(19) United States
(12) Patent Application Publication
(10) Pub. No.: US 2026/0325200 A1
(43) Pub. Date: Oct. 22, 2026

(54) HIGH INTENSITY FOCUSED ULTRASOUND AND BIPOLAR RADIOFREQUENCY COMBINED OUTPUT DEVICE WITH SUCTION FUNCTION

(71) Applicant: K1MED GLOBAL INC., Seoul (KR)

(72) Inventor: Sangsik KIM, Seoul (KR)

(73) Assignee: K1MED GLOBAL INC., Seoul (KR)

(21) Appl. No.: 19/529,138

(22) Filed: Feb. 4, 2026

Related U.S. Application Data
(63) Continuation of application No. PCT/KR2024/013854, filed on Sep. 12, 2024.

(30) Foreign Application Priority Data
Sep. 9, 2024 (KR) 10-2024-0122491

(51) Int. Cl. A61B 18/14; A61B 17/00; A61B 18/00; A61B 18/12; A61N 7/00; A61N 7/02

(52) U.S. Cl. CPC A61B 18/14; A61B 18/1206; A61N 7/02; A61B 2017/00566; A61B 2018/00464; A61B 2018/0091; A61B 2018/00994; A61B 2018/1467; A61N 2007/0008; A61N 2007/0034

(57) ABSTRACT

A high intensity focused ultrasound and bipolar radiofrequency combined output device with a suction function, includes a cylindrical housing that has an installation space therein and includes a body part at a front end portion thereof, a high intensity focused ultrasound (HIFU) output module that is provided in the housing, and a bipolar radiofrequency (Bipolar RF) output module that is provided in the housing to generate a bipolar radiofrequency in front of the housing using at least a pair of electrodes to which an electrical signal is applied, in which the body part includes a first body portion that is recessed backward to form a suction groove in a cup shape, and a second body portion that is formed to be extended outward from the first body portion to provide an annular surface.

### CROSS REFERENCE TO PRIOR APPLICATIONS

This application is a continuation application of PCT International Patent Application No. PCT/KR2024/013854 filed on Sep. 12, 2024, which claims priority to Korean Patent Application No. 10-2024-0122491 filed on Sep. 9, 2024, which are all hereby incorporated by reference in their entirety.

### BACKGROUND

The present invention relates to a device capable of suctioning a skin contact portion and then simultaneously providing high intensity focused ultrasound and bipolar radiofrequency output.

A high intensity focused ultrasound (HIFU) device is a device that uses a technique for focusing ultrasound energy on a specific depth of tissue in a non-invasive manner, and is widely used for therapeutic and cosmetic purposes. The HIFU device focuses ultrasound waves on a target portion of the skin to generate heat, thereby treating the target portion.

The high intensity focused ultrasound device may adjust the depth and intensity of the focused ultrasound waves according to the therapeutic purpose, and may be used on various depths of tissues such as the skin, a fat layer, and a muscle layer.

For example, the high intensity focused ultrasound (HIFU) may stimulate collagen production by applying heat to a dermal layer or a superficial musculo-aponeurotic system (SMAS), thereby providing the effect of improving wrinkles and increasing skin elasticity, or may focus heat on a subcutaneous fat layer to destroy fat cells, so that the destroyed fat cells are naturally excreted from the body, thereby providing the effect of improving a body shape.

In this regard, Korean Laid-open Patent Publication No. 10-2016-0053552 (hereinafter, referred to as ‘Prior Art 1’) discloses a high intensity focused ultrasound generating device capable of controlling a focal distance to form a thermal lesion by high intensity focused ultrasound.

A bipolar radiofrequency (RF) device generates radiofrequency current between two electrodes using electrical energy, and transmits thermal energy to tissues by the radiofrequency current, which is mainly used for collagen regeneration, lifting, and improvement of skin elasticity.

Specifically, when the radiofrequency current passes through the tissues, heat generated by resistance acts on a dermal layer of the skin to regenerate collagen and contract the skin, thereby providing a lifting effect.

In this regard, Korean Laid-open Patent Publication No. 10-2022-0155220 (hereinafter, referred to as ‘Prior Art 2’) discloses a radiofrequency current output device enabling sequential output of a monopolar type current and a bipolar type current or continuous output of the monopolar type current and the bipolar type current with a single operation.

However, in existing high intensity focused ultrasound output devices such as Prior Art 1, or existing bipolar radiofrequency output devices such as Prior Art 2, there are problems in that they are operated independently to simply provide independent treatment effects, making it difficult to provide integrated and balanced treatment due to unique limitations of the respective devices, and achieve the efficiency of treatment. That is, it is difficult to achieve synergy through cooperation of the respective devices.

### SUMMARY

The present invention has been made in an effort to solve the above-described problems associated with prior art, and it is an aspect to provide a technique capable of operating functions through high intensity focused ultrasound output and bipolar radiofrequency output together, as well as a suction function to pull the skin, thereby achieving synergy between the functions.

According to an aspect of the present invention, the aspect can be accomplished by the provision of a high intensity focused ultrasound and bipolar radiofrequency combined output device with a suction function, including a cylindrical housing that has an installation space therein and includes a body part at a front end portion thereof, a high intensity focused ultrasound (HIFU) output module that is provided in the housing to generate high intensity focused ultrasound in front of the housing based on an electrical signal through a piezoelectric ceramic member, and a bipolar radiofrequency (Bipolar RF) output module that is provided in the housing to generate a bipolar radiofrequency in front of the housing using at least a pair of electrodes to which an electrical signal is applied, in which the body part includes a first body portion that is recessed backward to form a suction groove in a cup shape, and is configured so that a front end portion of the high intensity focused ultrasound output module protrudes forward from the center of the suction groove, and a second body portion that is formed to be extended outward from the first body portion to provide an annular surface, on which the electrodes of the bipolar radiofrequency output module are uniformly arranged radially around the front end portion of the high intensity focused ultrasound output module to be exposed forward.

The body part may further include a suction hole providing portion having a tubular structure that protrudes backward from at least one position of the first body portion and is provided therein with a suction hole that connects the suction groove and the inside of the housing, and the high intensity focused ultrasound and bipolar radiofrequency combined output device may further include a suction module that is connected to the suction hole providing portion and provides suction force so that an annular area between a contact area of the high intensity focused ultrasound output module and a contact area of the electrodes of the bipolar radiofrequency output module on the skin is pulled back into the suction groove.

The front end portion of the high intensity focused ultrasound output module may be provided to protrude forward by a predetermined height with reference to a front side of the electrodes of the bipolar radiofrequency treatment module.

The high intensity focused ultrasound and bipolar radiofrequency combined output device may include a control module that is provided on one side of the housing to be electrically connected to the high intensity focused ultrasound output module, the bipolar radiofrequency output module, and the suction module and controls operations of the respective modules.

The control module may operate the suction module to allow the annular area between the contact area of the high intensity focused ultrasound output module and the contact area of the electrodes of the bipolar radiofrequency output module on the skin to be pulled back into the suction groove, and then, operate the high intensity focused ultrasound output module to output the high intensity focused ultrasound to a front central side and operate the bipolar radiofrequency output module to output the bipolar radiofrequency to a front peripheral side.

The control module may control, in operating the high intensity focused ultrasound output module to output the high intensity focused ultrasound, the high intensity focused ultrasound output module to continuously maintain the operation to generate the output in the form of a continuous line in accordance with an operational movement of the housing.

According to the present invention, the following effects are achieved.

First, through combined output of the high intensity focused ultrasound (HIFU) and the bipolar radiofrequency (Bipolar RF), it is possible to simultaneously achieve fat reduction and skin lifting, thereby preventing skin sagging and improving skin elasticity with one treatment.

Second, by adding the suction function, it is possible to pull back the skin and subcutaneous tissues to intensively deliver the ultrasound and radiofrequency energy thereto, thereby delivering energy efficiently to the tissues to maximize the treatment effect while reducing side effects.

Third, through simultaneous operations of the high intensity focused ultrasound (HIFU), bipolar radiofrequency (Bipolar RF), and suction functions, it is possible to enhance the synergy therebetween, thereby maximizing the fat reduction effect.

Fourth, it is possible to perform an integrated treatment for fat reduction and skin lifting, thereby shortening the treatment time.

Fifth, in the high intensity focused ultrasound output, it is possible to form a linear stimulation area by maintaining a continuous output, unlike the existing discontinuous output.

### BRIEF DESCRIPTION OF THE DRAWINGS

FIG. 1 is a perspective view showing a configuration of a high intensity focused ultrasound and bipolar radiofrequency combined output device according to an embodiment of the present invention.

FIG. 2 is a front view of the configuration of the high intensity focused ultrasound and bipolar radiofrequency combined output device according to the embodiment of the present invention.

FIG. 3 is a side view of the configuration of the high intensity focused ultrasound and bipolar radiofrequency combined output device according to the embodiment of the present invention.

FIG. 4 is a sectional view showing a configuration of main parts on a front side of the high intensity focused ultrasound and bipolar radiofrequency combined output device according to the embodiment of the present invention.

FIG. 5 is a diagram illustrating an output form of a high intensity focused ultrasound output module in the high intensity focused ultrasound and bipolar radiofrequency combined output device according to the embodiment of the present invention.

### DETAILED DESCRIPTION

Reference will hereinafter be made to various embodiments of the present invention, examples of which are illustrated in the accompanying drawings and described below, and the already known technical aspects will be omitted or briefly described for ease of description.

### 1. High Intensity Focused Ultrasound and Bipolar Radiofrequency Combined Output Device

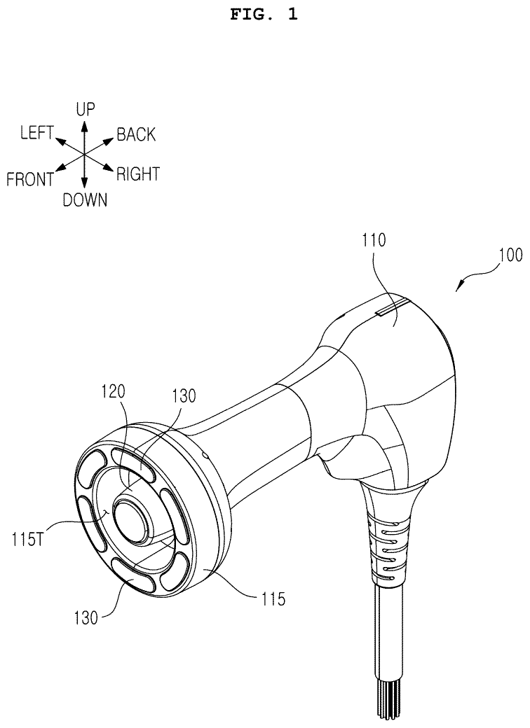

Referring to FIGS. 1 to 3, a high intensity focused ultrasound and bipolar radiofrequency combined output device 100 with a suction function, according to an exemplary embodiment of the present invention, is a device that may selectively or in combination utilize a high intensity focused ultrasound (HIFU) output-based function and a bipolar radiofrequency (Bipolar RF) output-based function, which includes a housing 110, a high intensity focused ultrasound output module 120, a bipolar radiofrequency output module 130, a suction module 140, and a control module 150.

The housing 110 is provided in a cylindrical shape having a predetermined installation space therein, and corresponds to an overall outer body that is directly held by an operator's hand to perform treatment.

Here, the housing 110 is provided, at a front end portion thereof, with an installation body part 115 that is configured to specifically define configurations of the high intensity focused ultrasound output module 120, the bipolar radiofrequency output module 130, and the suction module 140.

Specifically, the installation body part 115 includes a first body portion 115a that defines a front configuration of the high intensity focused ultrasound output module 120 and the suction module 140, a second body portion 115b that defines a front configuration of the bipolar radiofrequency output module 130, and a suction hole providing portion 115c that is formed for connection with the suction module 150.

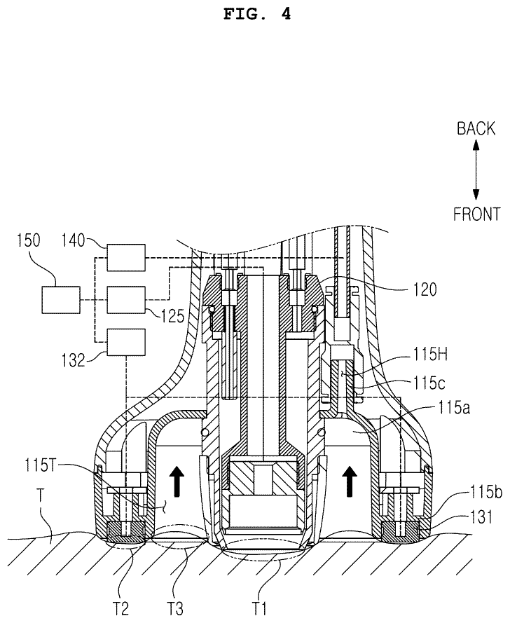

As shown in FIGS. 1 and 4, the first body portion 115a is recessed backward to form a suction groove 115T in a cup shape, and is configured so that a front end portion of the high intensity focused ultrasound output module 120 protrudes forward from the center of the suction groove 115T.

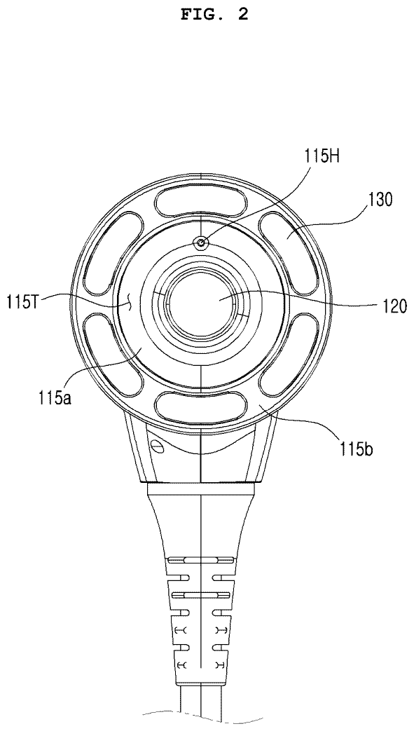

In addition, as shown in FIG. 2, the second body portion 115b is extended outward from the first body portion 115a to provide an annular installation surface, on which multiple electrodes 131 of the bipolar radiofrequency output module 130 are uniformly arranged radially around the front end portion of the high intensity focused ultrasound output module 120 to be exposed forward.

Preferably, the multiple electrodes 131 of the bipolar radiofrequency output module 130 are radially arranged in the annular form with equal intervals on the installation surface of the second body portion 115b to form at least one pair, preferably, three or four pairs, as shown in FIG. 2, so that the number thereof is 6 or 8.

The suction hole providing portion 115c having a tubular structure that protrudes backward from at least one position of the first body portion 115a and is provided therein with a suction hole 115H that connects the suction groove 115T and the inside of the housing 110.

Here, a rear portion of the suction hole providing portion 115c is connected to a front portion of a suction tube, and a rear portion of the suction tube is connected to the suction module 140 (which will be described later).

As shown in FIG. 4, the high intensity focused ultrasound output module 120 generates high intensity focused ultrasound (HIFU) at the front of the housing 10 based on an electrical signal through a piezoelectric ceramic member.

Specifically, a hemispherical or spherical cap-shaped piezoelectric ceramic member may be provided on the inner front side of the high intensity focused ultrasound output module 120 to generate high intensity focused ultrasound (HIFU) and provide an ultrasound stimulation area T1 formed within a predetermined range around a single focus at the front side.

First, a high intensity focused ultrasound output part 125 provided in the high intensity focused ultrasound output module 120 generates a radiofrequency AC electrical signal and applies the electrical signal to the piezoelectric ceramic member, and generates, during a process of converting energy of the electrical signal into mechanical energy in the form of vibration, ultrasound energy of waves in a radiofrequency band (mainly 1 to 7 MHz).

Accordingly, the ultrasound generated from the piezoelectric ceramic member is transmitted to a lens or a curved transducer to be focused within the specific area T1. This focusing may cause a thermal lesion through heat generation at the area T1.

As a result, in the portion where the ultrasound is focused, the mechanical energy of the ultrasound is absorbed by tissues to generate frictional heat, and this heat causes high temperature only in a specific depth of the focused area without affecting surrounding tissues, thereby destroying fat cells or connective tissues in the area.

In addition, the control module 150 controls the operation of the high intensity focused ultrasound output part 125 to adjust amplitude and frequency of the radiofrequency AC electrical signal applied to the piezoelectric ceramic member, thereby changing the intensity and focusing position of the ultrasound energy.

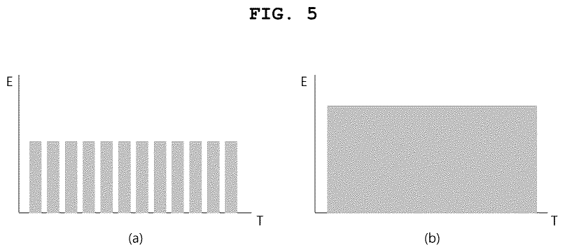

Preferably, the control module 150 controls, in operating the high intensity focused ultrasound output module 120 to output the high intensity focused ultrasound to a front central side, the high intensity focused ultrasound output module 120 to continuously maintain the operation to thereby generate the output in the form of a continuous line in accordance with an operational movement of the housing 110. The existing high intensity focused ultrasound (HIFU) device is operated in a like-linear method, where the device is switched on/off depending on frequencies to quickly take ultrasonic stimulations in a discontinuous (dot-like) manner as shown in FIG. 5A. In this method, as the module moves, dots are connected to form a linear stimulation area. However, this method has problems in discontinuous stimulation during the process of individually taking the dots and inconsistency in energy transmission.

Accordingly, as shown in FIG. 5B, the high intensity focused ultrasound output module 120 according to the present embodiment maintains a continuous ultrasonic output in conjunction with the control module 150. Thus, the high intensity focused ultrasound output module 120 continuously outputs the ultrasound energy in a real linear method without interruption while the housing 110 changes its posture and changes its output area to form a linear stimulation area, thereby ensuring consistent energy transmission and effective treatment effects along with shortened treatment time.

Specifically, the high intensity focused ultrasound output module 120 maintains the continuous ultrasound output, and allows the output ultrasound to form a continuous linear stimulation area even with the output position change due to the posture change of the housing 110, using the control module 150.

In this regard, in order to efficiently manage heat accumulated due to the continuous ultrasound output in the piezoelectric ceramic member to cause overheating, the high intensity focused ultrasound output module 120 of the present embodiment may include an additional component that circulates cooling water inside the device to absorb heat generated by the piezoelectric ceramic member, and discharges the cooling water to the outside to thereby efficiently dissipate the heat.

To this end, a water cooling part (not shown) may be provided around the piezoelectric ceramic member at the front portion of the high intensity focused ultrasound output module 120 to prevent a surrounding area of the piezoelectric ceramic member at the front portion of the high intensity focused ultrasound output module 120 from being overheated while enabling continuous ultrasound output to ensure stable and safe treatment.

The bipolar radiofrequency output module 130 generates bipolar radiofrequency (Bipolar RF) at the front portion of the housing 110 using at least one pair of electrodes 131 to which an electrical signal is applied.

To this end, the bipolar radiofrequency output module 130 includes the electrodes 131 that are radially arranged on the second body portion 115b, and a bipolar radiofrequency output part 132 that is electrically connected to the electrodes 131. The electrodes 131 and the bipolar radiofrequency output part 132 transmit alternating current in a radiofrequency range (200 to 1200 KHz). Friction energy caused by vibration of tissue ions generated by application of the alternating current to contact surfaces of the electrodes 131 increases the temperature of the tissues to form the radiofrequency stimulation area T2 that induces coagulation necrosis

Here, six or eight electrodes 131, that is, three or four pairs of electrodes so that the radiofrequency energy can flow from a positive electrode to the skin and return to an adjacent ground electrode.

As a result, the high intensity focused ultrasound and bipolar radiofrequency combined output device 100 has a configuration in which the front end portion of the high intensity focused ultrasound output module 120 protrudes at the front center of the housing 110, the cup-shaped suction groove 115T is provided in the first body portion 115a to surround the protruding front end of the high intensity focused ultrasound output module 120, and the multiple electrodes 131 of the bipolar radiofrequency output module 130 are radially arranged on the surface of the second body portion 115b on the outer side of the groove structure.

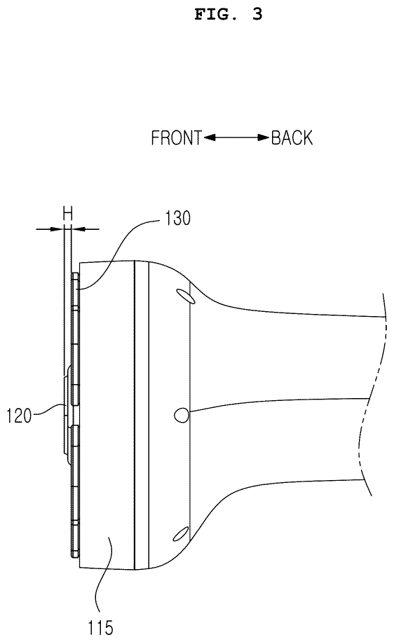

In addition, the front end portion of the high intensity focused ultrasound output module 120 is provided to protrude forward by a predetermined height (H) with reference to a front end portion of the electrodes 131 of the bipolar radiofrequency output module 130, as shown in FIG. 3.

Thus, it is possible to distinctly form the ultrasound stimulation area T1 based on the high intensity focused ultrasound (HIFU) (of the high intensity focused ultrasound output module 120, thereby ensuring that the ultrasound energy is focused efficiently to a target fat layer or dermal layer, without loss to the epidermis or other surrounding tissues, and deeply forming a suction area T3.

Furthermore, through the relative protrusion of the high intensity focused ultrasound output module 120, it is possible to allow the ultrasound waves to reach deeper layers safely without interfering with the skin surface where the radiofrequency stimulation area T2 is formed through the bipolar radiofrequency treatment module 130.

As shown in FIG. 4, the suction module 140 is connected to the suction hole providing portion 115c and provides suction force so that the annular area T3 between the contact area T1 of the high intensity focused ultrasound output module 120 and the contact area T2 of the plural electrodes 131 of the bipolar radiofrequency output module 130 on the skin T is pulled back into the suction groove 115T.

Specifically, the suction module 140 allows the skin to be pulled back into the suction groove 115T through suction, so that the skin tissue including an epidermal layer, a dermal layer, a superficial musculo-aponeurotic system layer, a subcutaneous fat layer, and a muscular layer can be lifted, to thereby move up even the muscular layer.

The control module 150 is provided on one side of the housing 110 to be electrically connected to the high intensity focused ultrasound output part 125 of the high intensity focused ultrasound output module 120, a bipolar radiofrequency output part 135 of the bipolar radiofrequency output module 130, and the suction module 140 and controls operations of the respective modules.

That is, the operations of the high intensity focused ultrasound output part 125 of the high intensity focused ultrasound output module 120, a bipolar radiofrequency output part 135 of the bipolar radiofrequency output module 130, and the suction module 140 are controlled by a control command generated by the control module 150 based on an input signal, so that functions of the high intensity focused ultrasound output, the bipolar radiofrequency output, and the suction can be performed simultaneously or selectively.

Preferably, the control module 150 may operate the suction module 140 to allow the area T3, which is the annular region between the contact area T1 of the high intensity focused ultrasound output module 120, and the contact area T2 of the electrodes 131 of the bipolar radiofrequency output module 130 on the skin to be pulled back into the suction groove 115T, and then, may operate the high intensity focused ultrasound output module 120 to output the high intensity focused ultrasound to the front central side and operate the bipolar radiofrequency output module 130 to output the bipolar radiofrequency to a front peripheral side.

### 2. Fat Reduction Effect Test Using High Intensity Focused Ultrasound and Bipolar Radiofrequency Combined Output Device

A test was performed to verify an actual clinical effect using the high intensity focused ultrasound and bipolar radiofrequency combined output device 100 according to the embodiment of the present invention, as described above. The test was performed by applying different control modes to the respective components by the control module 150 to observe a change level of fat reduction and skin elasticity before and after application of each control mode.

### Control Modes

Example 1: Simultaneous activation of all the high intensity focused ultrasound output module 120, the bipolar radiofrequency output module 130, and the suction module 140

Example 2: Activation of the high intensity focused ultrasound output module 120 and the bipolar radiofrequency output module 130

Example 3: Activation of the high intensity focused ultrasound output module 120 and the suction module 140

Example 4: Activation of the bipolar radiofrequency output module 130 and the suction module 140

### (2) Fat Reduction and Skin Elasticity Test

Five test targets (persons) were selected for each example, and a 10 cm×10 cm surface area on the front portion of the thigh was set as an output area to measure the thickness and elasticity of the thigh before using the device.

Then, the high intensity focused ultrasound output module 120 was set to uniformly maintain a frequency of 3 MHz and an energy intensity of 2.0 J/cm² as an output and the bipolar radiofrequency output module 130 was set to maintain the same output at a temperature of 40° C., and in this condition, the test was performed for 30 minutes a day for 7 days, and changes in the thigh thickness and elasticity were measured.

The skin elasticity change was measured by applying vibration stimuli to the skin surface using the DermaLab Elasticity Module (Cortex Technology) equipment based on the test method of ‘ISO 19399:2016’, and analyzing skin's deformation and restoration ability. Here, the skin elasticity change was measured in Pascals (Pa) according to how the skin reacts to applied pressure.

The changes in the thigh thickness and elasticity were obtained as an average of five test targets for each example, and the results are as follows (Table 1 and Table 2).

TABLE 1

| | Thickness | Change |
|---|---|---|
| Thigh Thickness | Change | Average |

| | | | | | |
|---|---|---|---|---|---|
| Example | Before | 60 | cm | −1.9 cm | −1.98 cm |
| 1 | After | 58.1 | cm | | |
| | Before | 62.8 | cm | −1.3 cm | |
| | After | 61.5 | cm | | |
| | Before | 54.7 | cm | −2.1 cm | |
| | After | 52.6 | cm | | |
| | Before | 75 | cm | −1.8 cm | |
| | After | 73.2 | cm | | |
| | Before | 65.8 | cm | −2.8 cm | |
| | After | 63 | cm | | |
| Example | Before | 61.5 | cm | −0.7 cm | −0.68 cm |
| 2 | After | 60.8 | cm | | |
| | Before | 57.5 | cm | −0.6 cm | |
| | After | 56.9 | cm | | |
| | Before | 65.2 | cm | −0.8 cm | |
| | After | 64.4 | cm | | |
| | Before | 72.2 | cm | −0.8 cm | |
| | After | 71.4 | cm | | |
| | Before | 68.2 | cm | −0.5 cm | |
| | After | 67.7 | cm | | |
| Example | Before | 61 | cm | −0.5 cm | −0.42 cm |
| 3 | After | 60.5 | cm | | |
| | Before | 57.3 | cm | −0.4 cm | |
| | After | 56.9 | cm | | |
| | Before | 62.5 | cm | −0.5 cm | |
| | After | 62 | cm | | |
| | Before | 74.6 | cm | −0.3 cm | |
| | After | 74.3 | cm | | |
| | Before | 66.8 | cm | −0.4 cm | |
| | After | 66.4 | cm | | |
| Example | Before | 59.1 | cm | −0.2 cm | −0.26 cm |
| 4 | After | 58.9 | cm | | |
| | Before | 63.7 | cm | −0.4 cm | |
| | After | 63.3 | cm | | |
| | Before | 69.1 | cm | −0.3 cm | |
| | After | 68.8 | cm | | |
| | Before | 72.4 | cm | −0.2 cm | |
| | After | 72.2 | cm | | |
| | Before | 75.2 | cm | −0.3 cm | |
| | After | 74.9 | cm | | |

TABLE 2

| | | |
|---|---|---|
| | Elasticity | Change |
| Skin elasticity | change | Average |

| | | | | |
|---|---|---|---|---|
| Example | Before | 256 Pa | 58 Pa | 58.2 Pa |
| 1 | After | 314 Pa | | |
| | Before | 274 Pa | 57 Pa | |
| | After | 331 Pa | | |
| | Before | 219 Pa | 45 Pa | |
| | After | 264 Pa | | |
| | Before | 281 Pa | 78 Pa | |
| | After | 359 Pa | | |
| | Before | 236 Pa | 53 Pa | |
| | After | 289 Pa | | |
| Example | Before | 256 Pa | 49 Pa | 47.8 Pa |
| 2 | After | 305 Pa | | |
| | Before | 274 Pa | 48 Pa | |
| | After | 322 Pa | | |
| | Before | 219 Pa | 38 Pa | |
| | After | 257 Pa | | |
| | Before | 281 Pa | 66 Pa | |
| | After | 347 Pa | | |
| | Before | 236 Pa | 38 Pa | |
| | After | 274 Pa | | |
| Example | Before | 256 Pa | 44 Pa | 42.2 Pa |
| 3 | After | 300 Pa | | |
| | Before | 274 Pa | 40 Pa | |
| | After | 314 Pa | | |
| | Before | 219 Pa | 34 Pa | |
| | After | 253 Pa | | |
| | Before | 281 Pa | 59 Pa | |
| | After | 340 Pa | | |
| | Before | 236 Pa | 34 Pa | |
| | After | 270 Pa | | |
| Example | Before | 236 Pa | 27 Pa | 30 Pa |
| 4 | After | 263 Pa | | |
| | Before | 281 Pa | 37 Pa | |
| | After | 318 Pa | | |
| | Before | 256 Pa | 35 Pa | |
| | After | 291 Pa | | |
| | Before | 284 Pa | 24 Pa | |
| | After | 308 Pa | | |
| | Before | 219 Pa | 27 Pa | |
| | After | 246 Pa | | |

As shown in Table 1, according to Example 1, in a state where the suction area T3 is pulled back into the suction groove 115T by simultaneous activation of the high intensity focused ultrasound output module 120, the bipolar radiofrequency output module 130, and the suction module 140 by the control module 150, in a case where the high intensity focused ultrasound is output to the front central side by the operation of the high intensity focused ultrasound output module 120 and the bipolar radiofrequency is output to the front peripheral side by the operation of the bipolar radiofrequency output module 130, the average of the thigh thickness change through fat reduction is ‘−1.98 cm’, and the reduction rate is about 3% to 4%, which shows a significant effect compared to the other examples.

As shown in Table 2, according to Example 1, in a state where the suction area T3 is pulled back into the suction groove 115T by simultaneous activation of the high intensity focused ultrasound output module 120, the bipolar radiofrequency output module 130, and the suction module 140 by the control module 150, in a case where the high intensity focused ultrasound is output to the front central side by the operation of the high intensity focused ultrasound output module 120 and the bipolar radiofrequency is output to the front peripheral side by the operation of the bipolar radiofrequency output module 130, the average of the skin elasticity change is ‘58.2 Pa’, and the improvement rate is about 22% to 23%, which shows a significant effect compared to the other examples.

As described above, according to the high intensity focused ultrasound and bipolar radiofrequency combined output device 100 having the above-described configurations and operations according to the embodiment of the present invention, it is possible to simultaneously provide the high intensity focused ultrasound output function and the bipolar radiofrequency output function while maintaining the suction function, using the high intensity focused ultrasound output module 120, the bipolar radiofrequency output module 130, and the suction module 140, and improving the synergy of the functions.

The embodiments disclosed in the present invention are not intended to limit the technical ideas of the present invention, but to describe the technical ideas, and the scope of the technical ideas of the present invention is not limited by the above-mentioned description. The scope of protection of the present invention should be defined by the appended claims, and all technical ideas within the scope of equivalents thereof should be construed as being included in the scope of the rights of the present invention.

### REFERENCE SIGNS

100: High intensity focused ultrasonic and bipolar radiofrequency composite output device
110: Housing
115: Body part
115a: First body portion
115b: Second body portion
115T: Suction groove
115c: Suction hole providing portion
115H: Suction hole
120: High intensity focused ultrasound output module
125: High intensity focused ultrasound output part
130: Bipolar radiofrequency output module
131: Electrode
132: Bipolar radiofrequency output part
140: Suction module
150: Control module

What is claimed is:

1. A high intensity focused ultrasound and bipolar radiofrequency combined output device with a suction function, comprising:
a cylindrical housing that has an installation space therein and includes a body part at a front end portion thereof;
a high intensity focused ultrasound (HIFU) output module that is provided in the housing to generate high intensity focused ultrasound in front of the housing based on an electrical signal through a piezoelectric ceramic member; and
a bipolar radiofrequency (Bipolar RF) output module that is provided in the housing to generate a bipolar radiofrequency in front of the housing using at least a pair of electrodes to which an electrical signal is applied;
wherein the body part comprises:
a first body portion that is recessed backward to form a suction groove in a cup shape, and is configured so that a front end portion of the high intensity focused ultrasound output module protrudes forward from the center of the suction groove; and
a second body portion that is formed to be extended outward from the first body portion to provide an annular surface, on which the electrodes of the bipolar radiofrequency output module are uniformly arranged radially around the front end portion of the high intensity focused ultrasound output module to be exposed forward.

2. The device according to claim 1, wherein the body part further comprises:
a suction hole providing portion having a tubular structure that protrudes backward from at least one position of the first body portion and is provided therein with a suction hole that connects the suction groove and the inside of the housing, and
wherein the high intensity focused ultrasound and bipolar radiofrequency combined output device further comprises:
a suction module that is connected to the suction hole providing portion and provides suction force so that an annular area between a contact area of the high intensity focused ultrasound output module and a contact area of the electrodes of the bipolar radiofrequency output module on the skin is pulled back into the suction groove.

3. The device according to claim 2, wherein the front end portion of the high intensity focused ultrasound output module is provided to protrude forward by a predetermined height with reference to a front side of the electrodes of the bipolar radiofrequency treatment module.

4. The device according to claim 2, further comprising:
a control module that is provided on one side of the housing to be electrically connected to the high intensity focused ultrasound output module, the bipolar radiofrequency output module, and the suction module and controls operations of the respective modules.

5. The device according to claim 4, wherein the control module operates the suction module to allow the annular area between the contact area of the high intensity focused ultrasound output module and the contact area of the electrodes of the bipolar radiofrequency output module on the skin to be pulled back into the suction groove, and then, operates the high intensity focused ultrasound output module to output the high intensity focused ultrasound to a front central side and operates the bipolar radiofrequency output module to output the bipolar radiofrequency to a front peripheral side.

6. The device according to claim 4, wherein the control module controls, in operating the high intensity focused ultrasound output module to output the high intensity focused ultrasound, the high intensity focused ultrasound output module to continuously maintain the operation to generate the output in the form of a continuous line in accordance with an operational movement of the housing.
